(19) United States
(12) Patent Application Publication
(10) Pub. No.: US 2015/0119726 A1
(43) Pub. Date: Apr. 30, 2015

(54) ELECTRONIC APPARATUS AND COMMUNICATION CONTROL METHOD

(71) Applicant: Kabushiki Kaisha Toshiba, Tokyo (JP)

(72) Inventors: Takaya Matsuno, Kunitachi Tokyo (JP); Takashi Sudo, Fuchu Tokyo (JP); Kentaro Takeda, Tokyo (JP); Yusaku Kikugawa, Ome Tokyo (JP); Shingo Suzuki, Sagamihara Kanagawa (JP); Yasuhiro Kanishima, Tokyo (JP)

(21) Appl. No.: 14/498,029

(22) Filed: Sep. 26, 2014

(30) Foreign Application Priority Data
Oct. 28, 2013 (JP) 2013-223222

(51) Int. Cl. A61B 5/00; A61B 5/0205

(52) U.S. Cl. CPC A61B 5/0022; A61B 5/02055; A61B 5/1118
USPC 600/483

(57) ABSTRACT

According to one embodiment, an electronic apparatus includes a sensor, an acquisition module, an estimation module, and a transmission controller. The sensor is configured to detect biomedical data of a user. The acquisition module is configured to acquire first and second biomedical data detected by the sensor. The estimation module is configured to estimate the user's status based on the first biomedical data. The transmission controller is configured to transmit the second biomedical data to an external apparatus at a timing in accordance with the user's estimated status.

### CROSS-REFERENCE TO RELATED APPLICATIONS

This application is based upon and claims the benefit of priority from Japanese Patent Application No. 2013-223222, filed Oct. 28, 2013, the entire contents of which are incorporated herein by reference.

### FIELD

Embodiments described herein relate generally to an electronic apparatus and a communication control method.

### BACKGROUND

In recent years, an electronic apparatus called a wearable device that a user can wear (put on) has become popular. A wearable device can take the form of a watch, a pair of glasses, a ring, a bracelet, a necklace, an adhesive plaster or the like. The user can acquire a variety of information via a display or speaker built in the wearable device that he or she wears.

A sensor to acquire various types of data of the user may be built in such a wearable device. This sensor can detect the user's data such as body temperature, pulse and acceleration (referred to as biomedical data hereinafter).

The biomedical data detected by a sensor built in a wearable device is, for example, transmitted to a server and then analyzed in the server. The analysis result of this biomedical data is offered to the user via the wearable device, allowing the user to acquire information based on the biomedical data of the user.

Since the above-mentioned wearable device is generally operated by a battery, a system to save power is necessary to allow the wearable device to operate for extended periods.

### BRIEF DESCRIPTION OF THE DRAWINGS

A general architecture that implements the various features of the embodiments will now be described with reference to the drawings. The drawings and the associated descriptions are provided to illustrate the embodiments and not to limit the scope of the invention.

FIG. 1 is an exemplary figure illustrating a network system comprising an electronic apparatus of a first embodiment.

FIG. 2 is an exemplary diagram illustrating a system structure of the electronic apparatus of the first embodiment.

FIG. 3 is an exemplary block diagram mainly illustrating a functional structure of the electronic apparatus of the first embodiment.

FIG. 4 is an exemplary flowchart illustrating a processing procedure of the electronic apparatus of the first embodiment.

FIG. 5 is an exemplary flowchart illustrating a processing procedure of an electronic apparatus of a second embodiment.

FIG. 6 is an exemplary block diagram mainly illustrating a functional structure of an electronic apparatus of a third embodiment.

FIG. 7 is an exemplary flowchart illustrating a processing procedure of the electronic apparatus of the third embodiment.

FIG. 8 is an exemplary figure illustrating a network system comprising an electronic apparatus of a fourth embodiment.

FIG. 9 is an exemplary block diagram mainly illustrating a functional structure of the electronic apparatus of the fourth embodiment.

FIG. 10 is an exemplary flowchart illustrating a processing procedure of the electronic apparatus of the fourth embodiment.

### DETAILED DESCRIPTION

In general, according to one embodiment, an electronic apparatus includes a sensor; an acquisition module; an estimation module; and a transmission controller. The sensor is configured to detect biomedical data of a user. The acquisition module is configured to acquire first and second biomedical data detected by the sensor. The estimation module is configured to estimate the user's status based on the first biomedical data. The transmission controller is configured to transmit the second biomedical data to an external apparatus at a timing in accordance with the user's estimated status.

Embodiments will be described hereinafter with reference to the accompanying drawings.

### First Embodiment

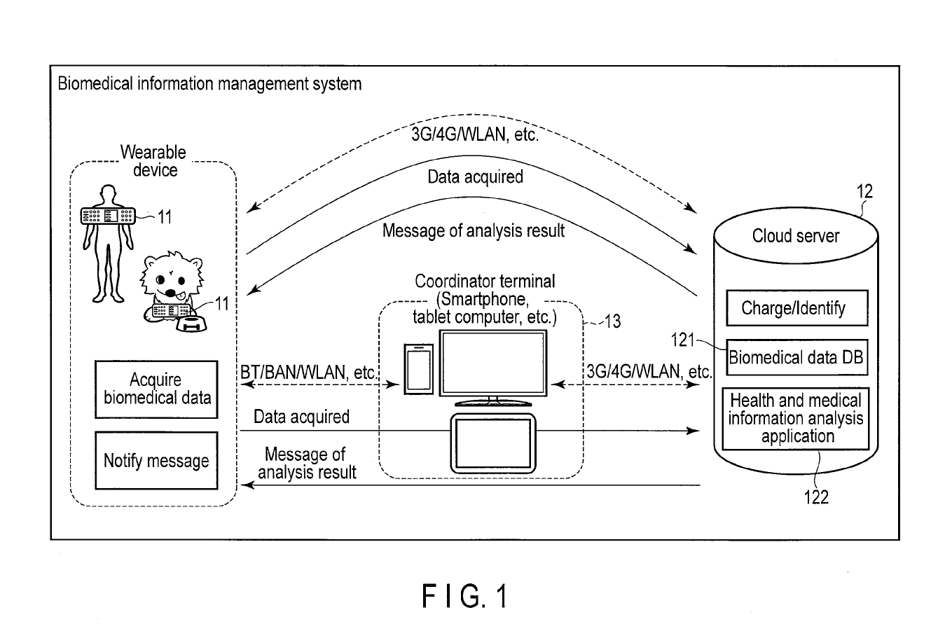

To begin with, a network system including an electronic apparatus of a first embodiment will be explained with reference to FIG. 1. In the following explanation, it is assumed that the network system is a biomedical information management system including a function to manage various types of biomedical data acquired by the electronic apparatus and to offer health information and medical information based on the biomedical data. Various types of biomedical data are detected by various types of sensor built in the electronic apparatus, including data of the user's body temperature, pulse and acceleration (user's biomedical data). Note that the electronic apparatus of the first embodiment can be realized as, for example, a wearable device 11 worn (put on) by the user.

The wearable device 11 can be realized as an embedded system built in various types of electronic apparatus. The wearable device 11 has a form such as a watch, a pair of glasses, a ring, a bracelet, a necklace and an adhesive plaster wearable for a human body. Note that the wearable device 11 can be attached to an animal as a collar or the like. In this case, the biomedical information management system can manage an animal's biomedical data.

The wearable device 11 acquires biomedical data by using various types of sensor built in the wearable device 11.

The wearable device 11 and a cloud server (referred to as a server hereinafter) 12 are communicably connected. In detail, connection is established between the wearable device 11 and the server 12 based on a wireless communication mode such as 3G mobile communication, 4G mobile communication and wireless LAN (WLAN).

This enables the wearable device 11 to transmit the acquired biomedical data to the server 12.

Also, the wearable device 11 can transmit biomedical data to the server 12 via a coordinator terminal 13 such as smartphone, tablet computer and television receiver. Connection is established between the wearable device 11 and the coordinator terminal 13 based on a wireless communication mode such as Bluetooth (registered trademark), body area network (BAN) and wireless LAN. In addition, connection is established between the coordinator terminal 13 and the server 12 based on a wireless communication mode such as 3G mobile communication, 4G mobile communication and wireless LAN (WLAN). This enables the biomedical data transmitted from the wearable device 11 to the coordinator terminal 13 to be transferred from the coordinator terminal 13 to the server 12.

The server 12 has a function to manage the wearable device 11 connected via a network. The server 12 identifies the wearable device 11 (or its user) requesting connection and executes processing for charging in accordance with service offering. Also, the server 12 receives biomedical data from the wearable device 11 and stores (accumulates) the received biomedical data in a biomedical data database (DB) 121 provided in the server 12.

Further, a health and medical information analysis application 122 is executed on the server 12. The health and medical information analysis application 122 transmits (notifies) a message (analysis result) such as health assistance and medical information to, for example, the wearable device 11 (or an administrator's terminal) by analyzing biomedical data stored in the biomedical data DB 121. This message includes information of heat stroke when the rise of the user's body temperature is detected. Note that the message can be transmitted to the wearable device 11 via the coordinator terminal 13.

The message transmitted by the server 12 is displayed on, for example, a display (screen) provided in the wearable device 11. This enables the user who wears the wearable device 11 to, for example, confirm his or her health status or acquire advice based on the health status. That is, the user can acquire simple information based on accumulated biomedical data before, for example, getting a diagnosis at a medical institution.

Note that while it will be explained below that the wearable device 11 and the server 12 directly transmit and receive data, it may also be possible that the wearable device 11 and the server 12 transmit and receive data via the coordinator terminal 13. The same holds true for the other embodiments explained below.

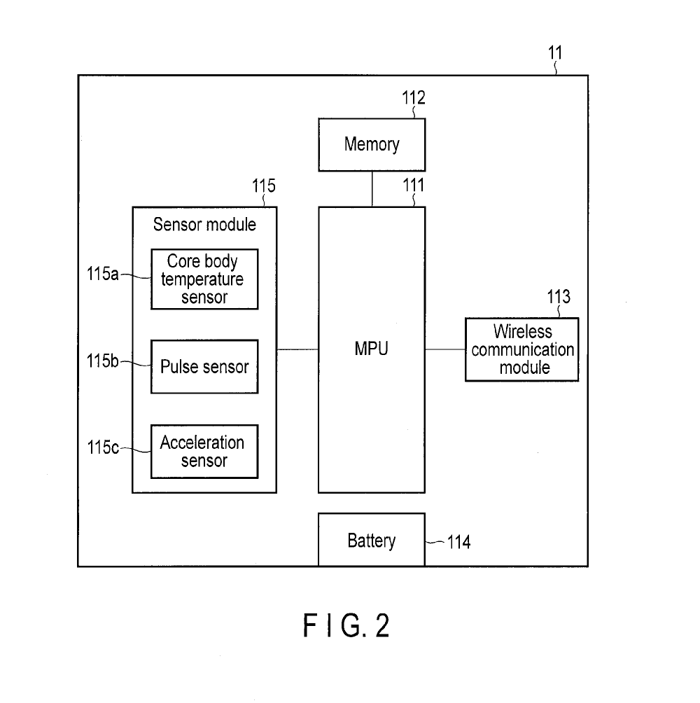

FIG. 2 is an exemplary diagram illustrating a system structure of the wearable device 11 shown in FIG. 1.

The wearable device 11 comprises an MPU 111, a memory 112, a wireless communication module 113, a battery 114 and a sensor module 115.

The MPU 111 is configured to control the operation of each component in the wearable device 11 and execute various types of software loaded into the memory 112. The software includes an operating system (OS) and a communication control program to control wireless communication in the wearable device 11. The communication control program is a program to realize a function to transmit various types of data including biomedical data by using the wireless communication module 113 and to receive a message transmitted from the server 12.

The wireless communication module 113 is configured to execute wireless communication with the server 12 by a wireless communication mode such as 3G mobile communication, 4G mobile communication and wireless LAN (WLAN). Note that the wireless communication module 113 is configured to execute wireless communication by a wireless communication mode such as Bluetooth, body area network (BAN) and wireless LAN when communicating with the coordinator terminal 13.

The battery 114 is configured to supply electricity to each component in the wearable device 11.

The sensor module 115, for example, is configured to detect the user's biomedical data. The sensor module 115 includes a core body temperature sensor 115a to detect biomedical data of the user's body temperature (body temperature data), a pulse sensor 115b to detect biomedical data of the user's pulse (pulse data) and an acceleration sensor 115c to detect biomedical data of acceleration of the user (or the wearable device 11 worn by the user) (acceleration data).

Note that biomedical data detected by the sensor module 115, called raw data, is output to the MPU 111 via an analogue front end (AFE) circuit, which is an analogue circuit that connects various types of sensor and the MPU 111.

The wearable device 11 further includes a display (display apparatus) configured to display a message or the like notified by the above-mentioned server 12, although not shown in FIG. 2.

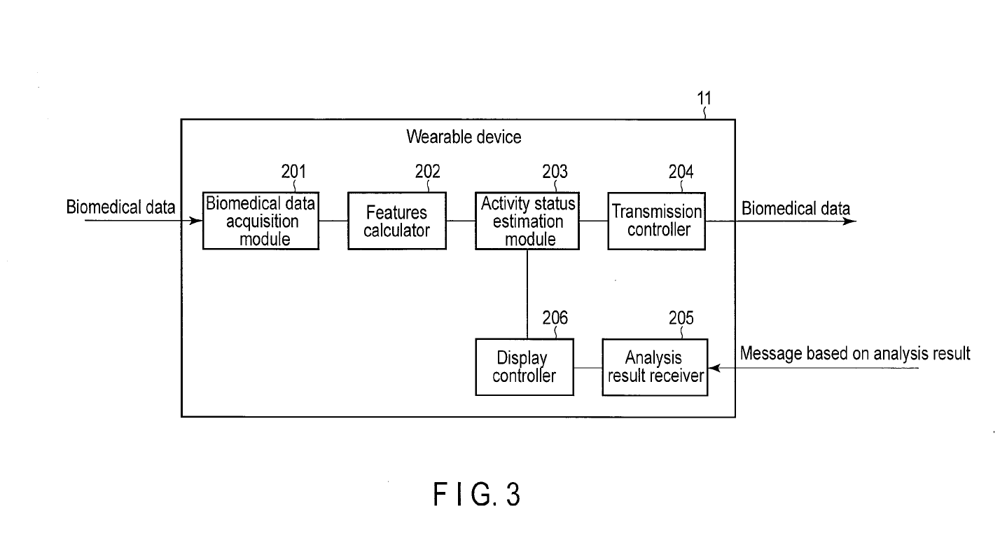

FIG. 3 is an exemplary block diagram mainly illustrating a functional structure of the wearable device 11 of the first embodiment. As shown in FIG. 3, the wearable device 11 includes a biomedical data acquisition module 201, a features calculator 202, an activity status estimation module 203, a transmission controller 204, an analysis result receiver 205 and a display controller 206, etc.

It is assumed in the first embodiment that each of these modules are realized by a communication control program which is loaded into the memory 112 and is executed by the MPU 111 shown in FIG. 2.

The biomedical data acquisition module 201 is configured to acquire biomedical data (raw data) detected by each of the sensors 115a-c included in the sensor module 115 shown in FIG. 2.

The features calculator 202 is configured to extract (calculate) features from biomedical data acquired by the biomedical data acquisition module 201. Note that features calculated by the features calculator 202 is smaller than biomedical data (raw data) acquired by the biomedical data acquisition module 201.

The activity status estimation module 203 is configured to estimate the activity status of a user who wears the wearable device 11 based on the features calculated based on biomedical data acquired by the biomedical data acquisition module 201 as mentioned above. It is assumed in the first embodiment that the user's activity status estimated by the activity status estimation module 203 includes “Sleeping,” “Moving” and “Exercising.”

The transmission controller 204 is configured to transmit to the server 12 (external apparatus) biomedical data acquired by the biomedical data acquisition module 201. At this time, the transmission controller 204 controls a timing (interval) of transmitting biomedical data in accordance with the user's activity status estimated by the activity status estimation module 203.

The biomedical data transmitted by the transmission controller 204 is received by the server 12. The biomedical data received by the server 12 is stored in the biomedical data DB 121 provided in the server 12.

The above-mentioned health and medical information analysis application 122 executed on the server 12 analyzes biomedical data stored in the biomedical data DB 121 and generates a message including health assistance or medical information for the user based on the analysis result. The message generated in the server 12 is then transmitted to the wearable device 11.

The analysis result receiver 205 is configured to receive a message transmitted by the server 12. This message is a message based on the analysis result of biomedical data acquired in the above-mentioned wearable device 11, including information of heat stroke when the rise of the user's body temperature is detected.

The display controller 206 displays a message received by the analysis result receiver 205 on the screen of the display, allowing the user to confirm his or her health status or acquire advice based on the health status. Note that this message can be offered as audio by using a speaker, earphone or the like.

Also, it may be possible that the display controller 206 displays the user's activity status estimated by the activity status estimation module 203.

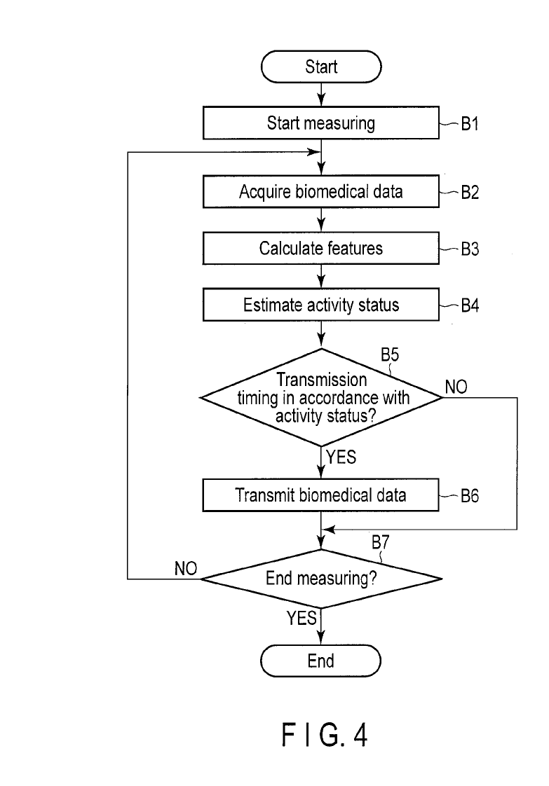

Next, the processing procedure of the wearable device 11 of the first embodiment will be explained with reference to a flowchart of FIG. 4. The processing of transmitting biomedical data acquired in the wearable device 11 to the server 12 will be mainly explained.

To begin with, the wearable device 11 starts measuring by the sensor module 115 (various types of sensor built in the wearable device 11) (block B1). The measurement by the sensor module 115 is started, for example, when the wearable device 11 is turned on. Note that the measurement by the sensor module 115 may be started in accordance with the instruction of a user who wears the wearable device 11.

The sensor module 115 includes the above-mentioned core body temperature sensor 115a, the pulse sensor 115b and the acceleration sensor 115c, etc. Note that the sensor module 115 may include a sensor capable of detecting biomedical data of, for example, the user's electrocardiogram and blood pressure, other than these sensors 115a-c.

When the measurement by the sensor module 115 is started, the biomedical data acquisition module 201 acquires biomedical data detected by the sensor module 115 (block B2). If the sensor module 115 includes the core body temperature sensor 115a, the pulse sensor 115b and the acceleration sensor 115c as mentioned above, biomedical data acquired by the biomedical data acquisition module 201 includes biomedical data of the user's body temperature (body temperature data), biomedical data of the user's pulse (pulse data) and biomedical data of the user's acceleration (acceleration data). In the text that follows, biomedical data (raw data) acquired by the biomedical data acquisition module 201 is called object biomedical data for explanation.

Next, the features calculator 202 calculates features from object biomedical data (block B3). For example, regarding pulse data detected by the pulse sensor 115b (biomedical data of the user's pulse), the features calculator 202 calculates a heart rate (features) by analyzing an amplitude that represents the pulse. While pulse data has been explained, the features calculator 202 calculates features for other biomedical data in a similar manner. This enables the features calculator 202 to acquire, for example, a body temperature, heart rate and acceleration, as features of object biomedical data.

Note that the features calculator 202 may improve reliability of object biomedical data by carrying out smoothing to object biomedical data before calculating features.

The activity status estimation module 203 estimates the activity status of a user who wears the wearable device 11 based on features (for example, body temperature, heart rate and acceleration) calculated by the features calculator 202 (block B4). It is assumed that the activity status estimation module 203 estimates that the user's activity status is, for example, “Sleeping,” “Moving” or “Exercising.”

The processing of estimating the user's activity status is executed by comparing, for example, features calculated by the features calculator 202 with a body temperature, heart rate and acceleration measured in advance in the case of “Sleeping,” “Moving” and “Exercising,” respectively. Note that if each of “Sleeping,” “Moving” and “Exercising” has not been measured in advance, it may be possible to compare with a value (body temperature, heart rate and acceleration) retained in advance in the wearable device 11.

The transmission controller 204 transmits the object biomedical data to the server 12 at a timing in accordance with the user's activity status estimated by the activity status estimation module 203. That is, the transmission controller 204 determines whether it is the transmission timing in accordance with the user's activity status estimated by the activity status estimation module 203 (block B5).

When the user is “Sleeping,” biomedical data is likely to change to a small extent. Therefore, when the user's activity status estimated by the activity status estimation module 203 is “Sleeping,” object biomedical data (or features) is transmitted to the server 12 only when, for example, the variation of the object biomedical data exceeds a threshold. That is, in the case where the user's activity status estimated by the activity status estimation module 203 is “Sleeping,” when the variation of object biomedical data exceeds a threshold, the transmission controller 204 determines that it is a timing of transmitting the object biomedical data in block B5. This makes it possible to widen the transmission interval of biomedical data and to reduce the number of times of transmitting biomedical data, since the object biomedical data is transmitted to the server 12 only when the variation of the object biomedical data exceeds a threshold.

Note that the variation of object biomedical data is calculated by storing biomedical data acquired one-time before the object biomedical data in, for example, a buffer (not shown) equipped with the wearable device 11 and by comparing the object biomedical data and the biomedical data. Also, it is assumed that a threshold compared with the variation of object biomedical data is a value calculated based on the variation that has been measured in advance when the user is “Sleeping,” in view of individual differences.

When the user is “Exercising,” biomedical data is likely to vary to a great extent (i.e., exponential variation is expected). Therefore, when the user's activity status estimated by the activity status estimation module 203 is “Exercising,” it is assumed that biomedical data is transmitted to the server 12 every time the biomedical data is acquired. That is, when the user's activity status estimated by the activity status estimation module 203 is “Exercising,” the transmission controller 204 determines that it is a timing of transmitting object biomedical data in block B5. This makes it possible to transmit as much biomedical data as is necessary for analysis in the server 12.

Also, when the user is “Moving,” it is likely that the variation of biomedical data is greater than when “Sleeping” but is smaller than when “Exercising.” Therefore, when the user's activity status estimated by the activity status estimation module 203 is “Moving,” it is assumed that biomedical data is transmitted to the server 12 less often than when the user's activity status is “Exercising” (for example, transmit biomedical data once, every time biomedical data is acquired twice). That is, in the case where the user's activity status estimated by the activity status estimation module 203 is “Moving,” when, for example, biomedical data acquired one-time before is not transmitted to the server 12, the transmission controller 204 determines that it is a timing of transmitting object biomedical data in block B5. This makes it possible to widen the transmission interval of biomedical data as compared with when the user's activity status is “Exercising,” and therefore reduce the number of times of transmitting the biomedical data.

As mentioned above, when the transmission controller 204 determines that it is a timing of transmitting object biomedical data (block B5, YES), the transmission controller 204 transmits the object biomedical data to the server 12 (block B6).

Note that object biomedical data transmitted to the server 12 is accumulated in the biomedical data DB 121 provided in the server 12. The biomedical data accumulated in the biomedical data DB 121 is analyzed by the health and medical information analysis application 122 performed on the server 12. The analysis result (the message based thereon) is transmitted to the wearable device 11 and is then displayed on the screen of the display of the wearable device 11.

On the other hand, when the transmission controller 204 determines that it is not a timing of transmitting object biomedical data (block B5, NO), the object biomedical data is not transmitted to the server 12 (i.e., the processing of block B6 is not executed).

Subsequently, it is determined whether or not the measurement by the sensor module 115 is ended (block B7). In detail, when a user who wears the wearable device 11 gives an instruction of ending measurement or turns off the wearable device 11, it is determined that the measurement by the sensor module 115 is ended.

When it is determined that the measurement by the sensor module 115 is not ended (block B7, NO), a step returns to block B2 mentioned above to repeat the processing. Note that it is assumed that the interval of acquiring biomedical data in the first embodiment is regular.

On the other hand, when it is determined that the measurement by the sensor module 115 is ended (block B7, YES), the processing is ended.

Note that when the transmission controller 204 determines that it is not a timing of transmitting object biomedical data in block B5, it is assumed that the object biomedical data (i.e., object biomedical data which is determined that it is not a transmission timing) is, for example, compressed to be accumulated in a buffer or the like. The biomedical data (biomedical data that has not been transmitted to the server 12) accumulated in a buffer can be transmitted to the server 12 together with object biomedical data at the next transmission timing. This makes it possible to avoid loss of biomedical data necessary for analysis in the server 12 as mentioned above and improve the accuracy of the analysis result.

Although it has been explained that biomedical data which is determined that it is not a transmission timing is transmitted at the next transmission timing, it may also be possible that the biomedical data is transmitted together when the amount of space of a buffer that accumulates biomedical data runs short or is not transmitted in view of power saving.

As mentioned above, the first embodiment is structured to acquire biomedical data detected by the sensor module 115 (each of the sensors 115a-c included therein), estimate the user's activity status based on the acquired biomedical data and transmit the biomedical data to the server 12 (external apparatus) at a timing in accordance with the user's estimated activity status. By dynamically controlling a timing (interval) of transmitting biomedical data to the server 12 in accordance with the user's activity status, such a structure enables the first embodiment to reduce the number of transmitting biomedical data. It is therefore possible to reduce consumption power and realize power saving in the wearable device 11.

While it has been explained in the first embodiment that biomedical data (raw data) acquired by the biomedical data acquisition module 201 is transmitted to the server 12, it may also be possible that features calculated by the features calculator 202 instead of the biomedical data is transmitted to the server 12. According to such a structure, since the amount of data is smaller in features calculated by the features calculator 202 than biomedical data (raw data), it is possible to reduce the amount of data transmitted to the server 12 and to further save power in the wearable device 11.

Also, while it has been explained in the first embodiment that the wearable device 11 and the server 12 directly receive and transmit data with each other, it may also be possible that data is transmitted and received via the coordinator terminal 13 as mentioned above. Note that when biomedical data is transmitted to the coordinator terminal 13 in such a case, it is possible to make a structure to control the operation of the coordinator terminal 13 by setting a flag indicating that the biomedical data is instantly transferring to the server 12 or is once accumulated in the coordinator terminal 13. This makes it possible to save power in the whole system as well as the wearable device 11.

Further, while it has been explained in the first embodiment that the user's activity status is estimated by the activity status estimation module 203 included in the wearable device 11, when the estimation processing of the user's activity status cannot be executed in the wearable device 11 because of, for example, low processing capacity of the MPU 111 or small amount of the memory 112 in the wearable device 11, it is possible that the processing is structured to be executed in an external apparatus (for example, the server 12). In this case, data (biomedical data) necessary for estimation processing of the user's activity status should be transmitted to the server 12 (or the coordinator terminal 13), and the server 12 should execute estimation processing of the user's activity status and transmission control processing of biomedical data in the wearable device 11.

### Second Embodiment

Next, a second embodiment will be explained. As with the above-mentioned first structure, it is assumed that the electronic apparatus of the second embodiment is, for example, realized as a wearable device worn by a user and included in the biomedical information management system shown in FIG. 1.

Also, the structure of the electronic apparatus (wearable device) of the second embodiment will be explained with reference to FIGS. 2 and 3, since it is the same as the structure of the first embodiment.

In the following text, while the same portions as the first embodiment will omit detailed explanation, the portions different from the first embodiment will be mainly mentioned.

The second embodiment differs from the above-mentioned first embodiment. While the first embodiment is structured to control a timing of transmitting biomedical data to the server 12 in accordance with the user's activity status, the second embodiment is structured to control a timing (interval) of acquiring biomedical data in accordance with the user's activity status.

That is, the biomedical data acquisition module 201 included in the wearable device 11 of the second embodiment acquires biomedical data (biomedical data detected by the sensor module 115) next to the biomedical data transmitted to the server 12, at a timing in accordance with the user's activity status estimated by the activity status estimation module 203.

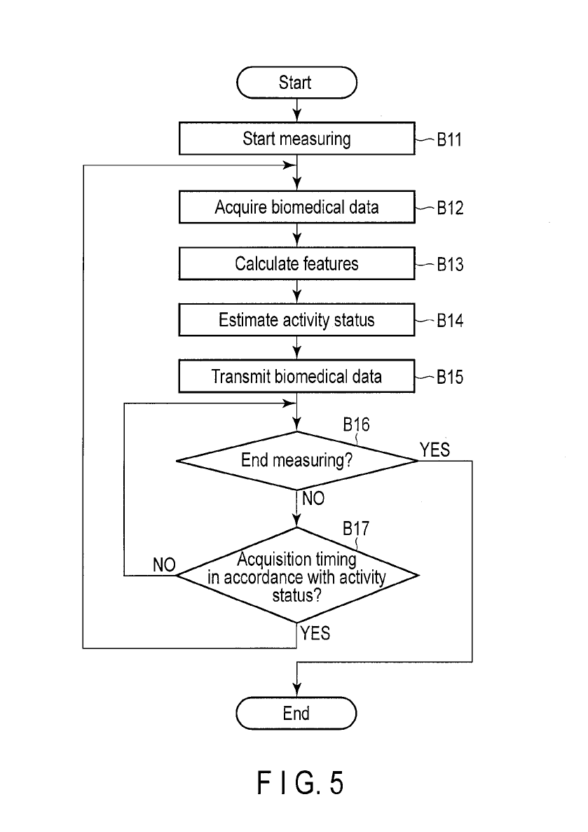

The processing procedure of the wearable device 11 of the second embodiment will be explained below with reference to a flowchart of FIG. 5. In the following, the processing of transmitting biomedical data acquired in the wearable device 11 to the server 12 will be mainly explained.

To begin with, the processing of blocks B11-14 corresponding to that of blocks B1-4 shown in FIG. 4 mentioned above is executed. In the following, biomedical data acquired by block B12 is called object biomedical data for explanation.

Next, the transmission controller 204 transmits object biomedical data to the server 12 (block B15).

After the processing of block B15 is executed, the processing of B16 corresponding to that of B7 shown in FIG. 4 mentioned above is executed.

When it is determined in block B16 that the measurement by the sensor module 115 is ended, the processing is ended.

On the other hand, when it is determined in block B16 that the measurement by the sensor module 115 is not ended, the biomedical data acquisition module 201 acquires the next biomedical data at a timing in accordance with the user's activity status estimated by the activity status estimation module 203. That is, the biomedical data acquisition module 201 determines whether it is the acquisition timing in accordance with the user's activity status estimated by the activity status estimation module 203 (block B17).

When the user is “Sleeping,” biomedical data is likely to change to a small extent. Therefore, when the user's activity status estimated by the activity status estimation module 203 is “Sleeping,” a period (referred to as a first period hereinafter) is set to make an acquisition interval longer than the acquisition interval of biomedical data when the after-mentioned user is awake (“Moving” and “Exercising”). That is, in the case where the user's activity status estimated by the activity status estimation module 203 is “Sleeping,” when the first period passes after object biomedical data is acquired in block B12, the biomedical data acquisition module 201 determines that it is a timing of acquiring the next biomedical data in block B17. This makes it possible to widen the acquisition interval of biomedical data as compared with when the user's activity status is “Moving” and “Exercising,” and therefore to reduce the number of times of transmitting object biomedical data.

When the user is “Exercising,” biomedical data is likely to change to a great extent. Therefore, when the user's activity status estimated by the activity status estimation module 203 is “Exercising,” a period (referred to as a second period hereinafter) is set to make an acquisition interval shorter than the acquisition interval of biomedical data when the above-mentioned user is “Sleeping”. That is, in the case where the user's activity status estimated by the activity status estimation module 203 is “Exercising,” when the second period passes after object biomedical data is acquired in block B12, the biomedical data acquisition module 201 determines that it is a timing of acquiring the next biomedical data in block B17. This makes it possible to shorten the acquisition interval of biomedical data as compared with when the user's activity status is “Sleeping,” and therefore to transmit as much biomedical data as is necessary for analysis in the server 12.

Also, when the user is “Moving,” it is likely that the variation of biomedical data is greater than when “Sleeping” but is smaller than when “Exercising.” Therefore, when the user's activity status estimated by the activity status estimation module 203 is “Moving,” a period (referred to as a third period hereinafter) is set to make an acquisition interval shorter than the acquisition interval when the above-mentioned user is “Sleeping” and longer than the acquisition interval when the above-mentioned user is “Exercising.” That is, in the case where the user's activity status estimated by the activity status estimation module 203 is “Moving,” when the third period passes after object biomedical data is acquired in block B12, the biomedical data acquisition module 201 determines that it is a timing of acquiring the next biomedical data in block B17. Since this makes it possible to secure biomedical data as much data as is necessary for analysis in the server 12 and to widen the acquisition interval of biomedical data as compared with when the user's activity status is “Exercising,” it is possible to reduce the number of transmitting object biomedical data.

As mentioned above, when the biomedical data acquisition module 201 determines that it is a timing of acquiring biomedical data (block B17, YES), a step returns to block B12 mentioned above to repeat the processing.

On the other hand, when the biomedical data acquisition module 201 determines that it is not a timing of acquiring biomedical data (block B17, NO), a step returns to block B16 to repeat the processing. That is, the next biomedical data is not acquired until the acquisition timing.

While it has been explained in FIG. 5 that biomedical data acquired in block B12 is transmitted after the processing of blocks B13 and B14 is executed, it may also be possible that a timing of transmitting biomedical data is further controlled, as explained in, for example, the first embodiment above.

As mentioned above, the second embodiment is structured to acquire biomedical data detected by the sensor module 115 (each of the sensors 115a-c included therein), estimate the user's activity status based on the acquired biomedical data, transmit the biomedical data to the server 12 (external apparatus) and acquire the biomedical data next to biomedical data transmitted to the server 12 at a timing in accordance with the user's activity status. By dynamically controlling a timing (interval) of acquiring biomedical data in accordance with the user's activity status, such a structure enables the second embodiment to reduce the number of transmitting the biomedical data. Therefore, it is possible to reduce consumption power and realize power saving in the wearable device 11.

### Third Embodiment

Subsequently, a third embodiment will be explained. As with the above-mentioned first and second embodiments, it is assumed that the electronic apparatus of the third embodiment is, for example, realized as a wearable device worn by a user and included in the biomedical information management system shown in FIG. 1.

The communication status in a communication channel by connection between a wearable device and a server (external apparatus) is likely to vary because of various factors. For example, when the communication status is good, it is possible to transmit a predetermined amount of data (for example, biomedical data) to the server 12 in a short time. That is, the consumption power is small in this case. On the other hand, when the communication status is not good, it takes longer to transmit a predetermined amount of data to the server 12. That is, the consumption power is large in this case.

For this reason, in the third embodiment, when the communication status is not good between a wearable device and the server 12, the priority of each sensor based on the user's activity status is calculated to transmit biomedical data to the server 12 based on the priority.

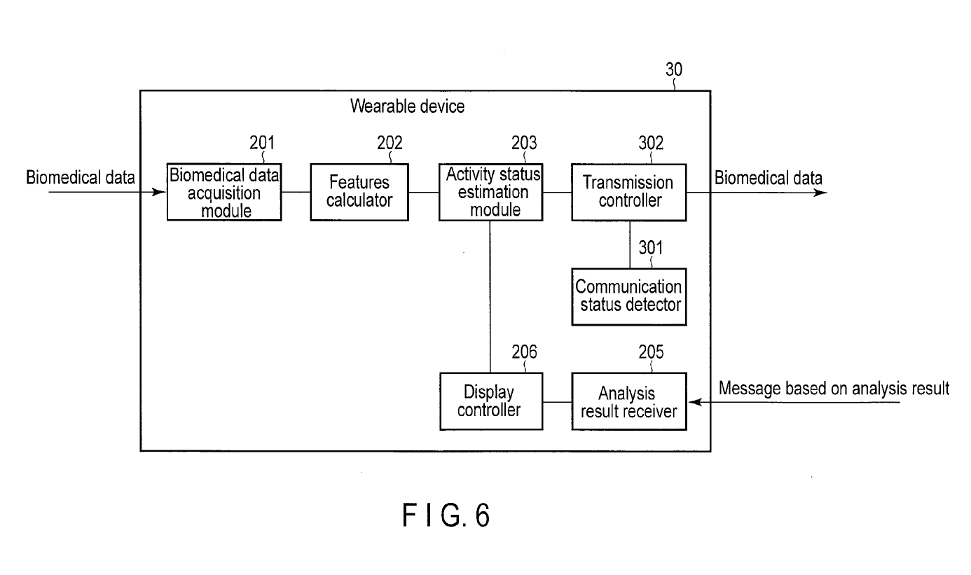

FIG. 6 is an exemplary block diagram mainly illustrating the functional structure of the electronic apparatus of the third embodiment. Note that the same portions as FIG. 3 mentioned above are put the same numeral references to omit detailed explanation. In the following text, the portions different from FIG. 3 will be mainly described.

Also, the system structure of the wearable device of the third embodiment is the same as that of the above-mentioned first and second embodiments and therefore will be explained with reference to FIG. 2.

As shown in FIG. 6, a wearable device 30 of the third embodiment includes a communication status detector 301 and a transmission controller 302.

The communication status detector 301 is configured to detect the communication status between the server 12 and the wireless communication module 113 included in the wearable device 30. In other words, the communication status detector 301 is configured to detect the status of the communication channel between the server 12 and the wireless communication module 113.

In detail, the communication status detector 301 is configured to calculate for the wireless communication module 113 an evaluation value of a communication status based on signal intensity of a signal received from the server 12 (i.e., radiowave intensity of received radiowave), response time and data transmission speed in data transmission and reception, and the like. It is assumed that, for example, a greater evaluation value indicates a better communication status.

The transmission controller 302 is configured to control a timing of acquiring biomedical data in accordance with the user's activity status, as explained in the second embodiment above.

Also, the transmission controller 302 is configured to control transmitting to the server 12 biomedical data acquired by the biomedical data acquisition module 201 in accordance with the communication status detected by the communication status detector 301.

In detail, when the communication status is not good (poor), the transmission controller 302 calculates the priority of each of the sensors 115a-c based on the user's activity status estimated by the activity status estimation module 203 and transmits biomedical data detected by a sensor in which the calculated priority is greater than a value set in advance, among the sensors 115a-115c.

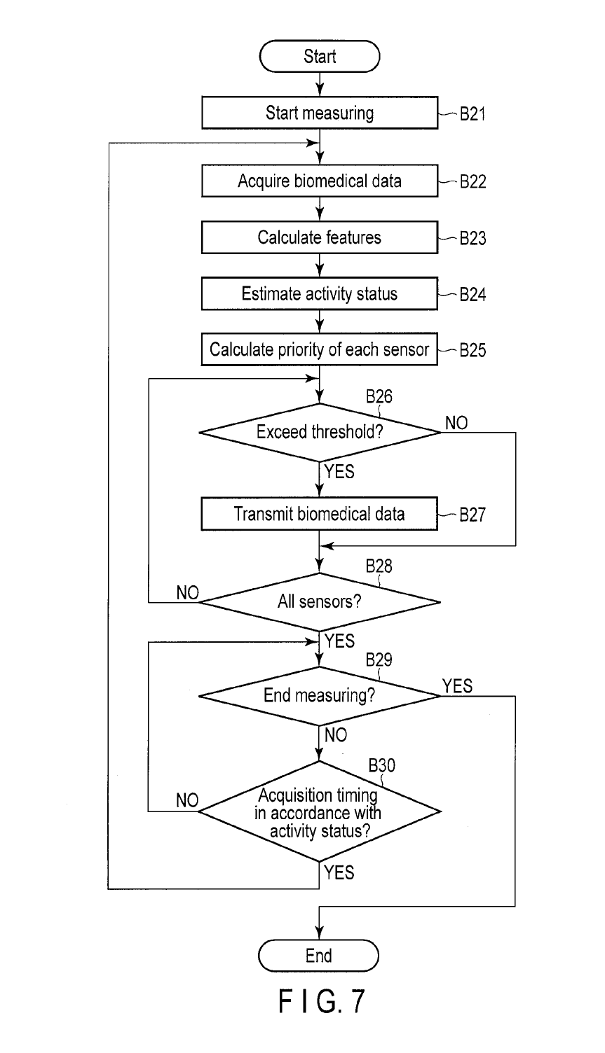

In the following, the processing procedure of the wearable device 30 of the third embodiment will be explained with reference to a flowchart of FIG. 7. The processing of transmitting to the server 12 biomedical data acquired in the wearable device 30 will be mainly described.

Note that FIG. 7 shows the processing procedure of the wearable device 30 executed when the communication status is not good between the wearable device 30 and the server 12.

The determination as to whether the communication status is good between the wearable device 30 and the server 12 is made based on the above-mentioned evaluation value of the communication status calculated by the communication status detector 301.

In detail, when the evaluation value of the communication status is below a value set in advance, it is determined that the communication status is not good between the wearable device 30 and the server 12, and the processing shown in FIG. 7 is executed.

On the other hand, when the evaluation value of the communication status exceeds a value set in advance, it is determined that the communication status is good between the wearable device 30 and the server 12. Note that when it is determined that the communication status is good between the wearable device 30 and the server 12, the processing of, for example, FIG. 5 as mentioned above is executed.

When it is determined that the communication status is not good between the wearable device 30 and the server 12 as mentioned above, the processing of blocks B21-B24 corresponding to that of B11-B14 is executed. In the following, biomedical data acquired in block B22 is called object biomedical data for explanation.

Next, the transmission controller 204 prioritizes each of the sensors 115a-c included in the sensor module 115 based on the user's activity status estimated by the activity status estimation module 203. In this case, the transmission controller 204 calculates the priority of each of the sensors 115a-c included in the sensor module 115 (block B25).

When the user is “Sleeping,” it is assumed that the transmission controller 204 calculates a high priority to the core body temperature sensor 115a and the pulse sensor 115c to control, for example, the user's body temperature and pulse. On the other hand, when the user is “Sleeping,” it is assumed that the transmission controller 204 calculates a low priority to the acceleration sensor 115c since the user's movement does not vary to a great extent.

When the user is “Exercising,” it is assumed that the transmission controller 204 calculates a high priority to the core body temperature sensor 115a, the pulse sensor 115b and the acceleration sensor 115c to observe the variation in the user's body temperature, pulse and acceleration.

Also, when the user is “Moving,” it is assumed that the transmission controller 204 calculates a high priority to the pulse sensor 115b to control the user's pulse. On the other hand, when the user is “Moving,” it is assumed that the transmission controller 204 calculates a low priority to the core body temperature sensor 115a and the acceleration sensor 115c since the user's temperature and movement do not vary to a great extent.

Note that the priority of each of the sensors 115a-c is calculated by, for example, weighting the priority retained in advance in the wearable device 30 in accordance with the user's activity status. Also, it may be possible that the method for calculating the priority of each of the sensors 115a-c is changed in accordance with the contents of service offered by the server 12.

While only the core body temperature sensor 115a, the pulse sensor 115b and the acceleration sensor 115c have been explained, other sensors will be explained briefly below. For example, when the user is “Exercising,” his or her heart can get high pressure. The sensor module 115 therefore includes an electrocardiographic sensor, which makes it possible to give a higher priority to the electrocardiographic sensor when the user's activity status estimated by the activity status estimation module 203 is “Exercising.” Also, when snoring of “Sleeping” or the like is observed, it is possible to give a higher priority to a microphone. Further, when there is a concern of having apnea in “Sleeping,” it is possible to give a higher priority to the pulse sensor 115b to measure a blood oxygen level.

Next, the transmission controller 204 executes the processing of blocks B26 and B27 to each of the sensors 115a-c included in the sensor module 115. In the following, a sensor subject to the processing of blocks B26 and B27 is called object sensor.

To begin with, the transmission controller 204 determines whether or not the priority of an object sensor calculated exceeds a value set in advance (referred to as a threshold hereinafter) (block B26).

When it is determined that the priority of an object sensor exceeds a threshold (block B26, YES), the transmission controller 204 transmits to the server 12 biomedical data detected by the object sensor, among biomedical data acquired by the biomedical data acquisition module 201 in block B22 mentioned above (block B27).

On the other hand, when the priority of an object sensor does not exceed a threshold (block B26, NO), the processing of block B27 is not executed. In other words, biomedical data detected by an object sensor whose priority does not exceed a threshold is not transmitted to the server 12.

Next, it is determined whether or not the processing of blocks B26 and B27 mentioned above has been executed for all the sensors (block B28).

When it is determined that the processing has not been executed for all the sensors (block B28, NO), a step returns to block B26 mentioned above to repeat the processing. In this case, a sensor for which the processing of blocks B26 and B27 has not been executed is treated as an object sensor, and the processing of B26 is executed.

On the other hand, when it is determined that the processing has been executed for all the sensors (block B28, YES), the processing of blocks B29 and B30 mentioned above corresponding to that of blocks B16 and B17 mentioned above in FIG. 5 is executed.

Note that biomedical data that has not been transmitted to the server 12 since its priority does not exceed a threshold may be accumulated in a buffer included in the wearable device 30 and be transmitted to the server 12 when the communication status is good. This makes it possible to avoid loss of the biomedical data necessary for analysis in the server 12 as mentioned above and improve the accuracy of the analysis result.

As mentioned above, the third embodiment is structured to calculate the priority of each of the sensors 115a-c based on the user's activity status in accordance with the status of communication with the server 12 and to transmit to the server 12 biomedical data detected by the sensor when the priority exceeds a threshold. Under such a structure in the third embodiment, in view of the fact that consumption power increases because of the increase in a transmission time when a communication status is not good (poor) as mentioned above, it is possible to transmit biomedical data detected by the high-priority sensor, to reduce the amount of data transmitted to the server 12 and therefore to save power when the communication status is not good.

While it has been explained that the above-mentioned processing shown in FIG. 7 is executed when the communication status is not good between the wearable device 30 and the server 12, the processing may also be executed, for example, when the remaining battery capacity of the wearable device 30 is less than a value set in advance. This makes it possible to prolong a continuous operation time by executing the processing shown in FIG. 7.

Also, when the communication status is not good between the wearable device 30 and the server 12, it may be possible that the communication status to transmit biomedical data is improved by switching of a wireless communication mode with the server 12 or switching to communication via the coordinator terminal 13. That is, it is possible that the processing shown in FIG. 7 mentioned above can be executed when the communication status is not improved even by taking these steps.

Further, while it has been explained that a timing of acquiring biomedical data is controlled in accordance with the user's activity status in FIG. 7, the third embodiment may be applied when controlling a timing of transmitting biomedical data to the server 12 in accordance with the user's activity status explained in the first embodiment above.

Furthermore, while it has been explained in the third embodiment that biomedical data (raw data) acquired by the biomedical data acquisition module 201 is transmitted to the server 12, it may be possible that features calculated by the features calculator 202 instead of the biomedical data is transmitted to the server 12. According to such a structure, since the amount of data transmitted to the server 12 can be reduced, it is possible to further save power in the wearable device 30.

### Fourth Embodiment

Next, a fourth embodiment will be explained. In the following explanation, it is assumed that the electronic apparatus of the fourth embodiment is realized as, for example, a wearable device worn by a user, as with the above-mentioned first, second and third embodiments.

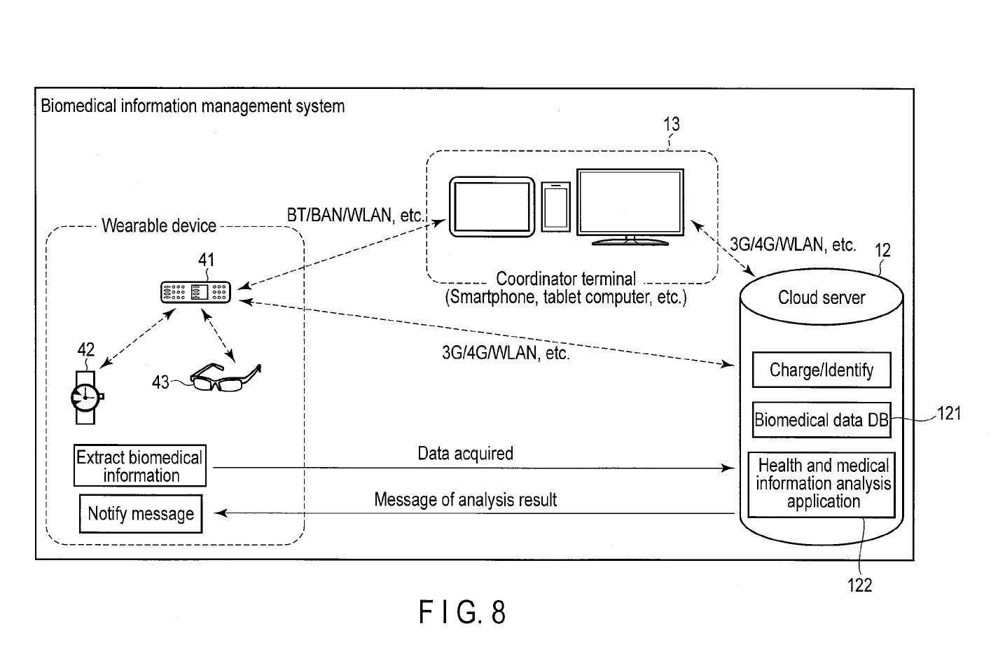

While the wearable device of the fourth embodiment is included in the biomedical information management system as with the above-mentioned first, second and third embodiments, it is assumed in the fourth embodiment that the user, for example, wears a plurality of wearable devices 41-43 that differ in processing capacity (performance) as shown in FIG. 8 and that data is transmitted from one of the wearable devices 41-43.

Note that a processing capacity is determined by various types of information (parameter) such as processing speed of a processor, memory size (buffer size), consumption power, remaining battery capacity and signal intensity.

Note in FIG. 8 that the same portions as FIG. 1 mentioned above are put the same numeral references to omit detailed explanation.

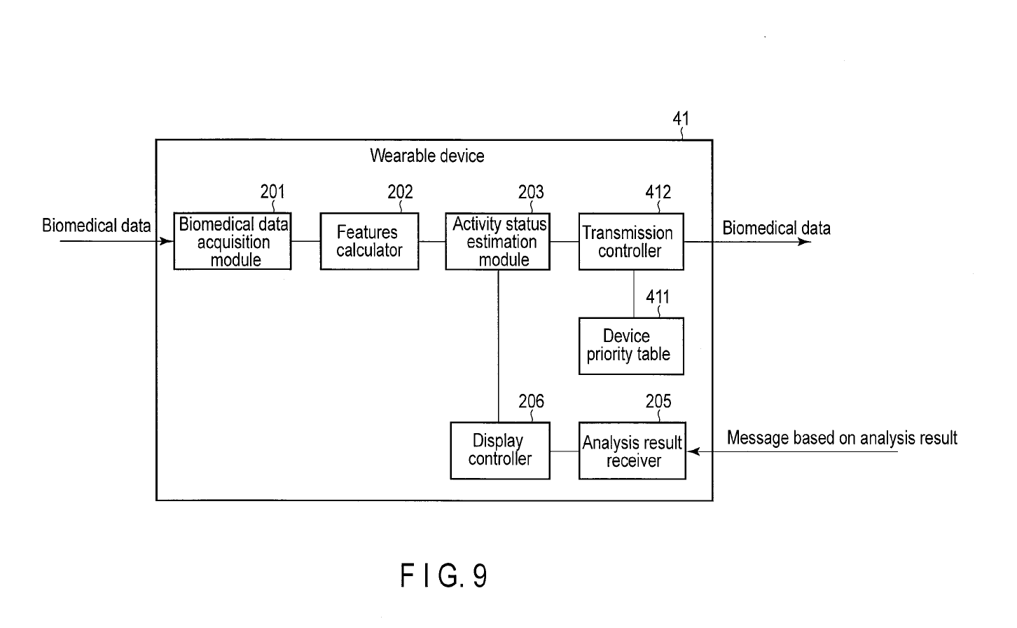

FIG. 9 is an exemplary block diagram mainly illustrating a functional structure of the electronic apparatus of the fourth embodiment. Note that the same portions as FIG. 3 mentioned above are put the same numeral references to omit detailed explanation. In the following, the portions that differ from FIG. 3 will be mainly explained. Regarding FIG. 9, the wearable device 41 will be explained among the wearable devices 41-43 worn by the user.

Also, the system structure of the wearable device of the fourth embodiment will be explained with reference to FIG. 2 since it is the same as the above-mentioned first, second and third embodiments.

As shown in FIG. 9, the wearable device 41 of the fourth embodiment includes a device priority table 411 and a transmission controller 412.

The device priority table 411 is configured to retain information of each processing capacity of the above-mentioned wearable devices 41-43 worn by the user (referred to as processing capacity information hereinafter). The processing capacity information includes information such as processing speed of a processor, memory size (buffer size), consumption power, remaining battery capacity and signal intensity as mentioned above.

The transmission controller 412 is configured to control a timing of acquiring biomedical data in accordance with the user's activity status, as explained in the above-mentioned second embodiment.

Also, the transmission controller 412 is configured to control transmitting biomedical data acquired by the biomedical data acquisition module 201 based on the processing capacity information retained in the device priority table 411.

In detail, the transmission controller 412 is configured to calculate the priority of each of the wearable devices 41-43 based on the processing capacity information retained in the device priority table 411 and transmit biomedical data to an external apparatus based on the calculated priority. Note that an external apparatus to which biomedical data is transmitted by the transmission controller 412 includes the server 12 (or the coordinator terminal 13) and the other wearable devices 42 and 43.

While the functional structure of the wearable device 41 in FIG. 9 has been explained, since the same holds true for the other wearable devices 42 and 43, its detailed explanation will be omitted.

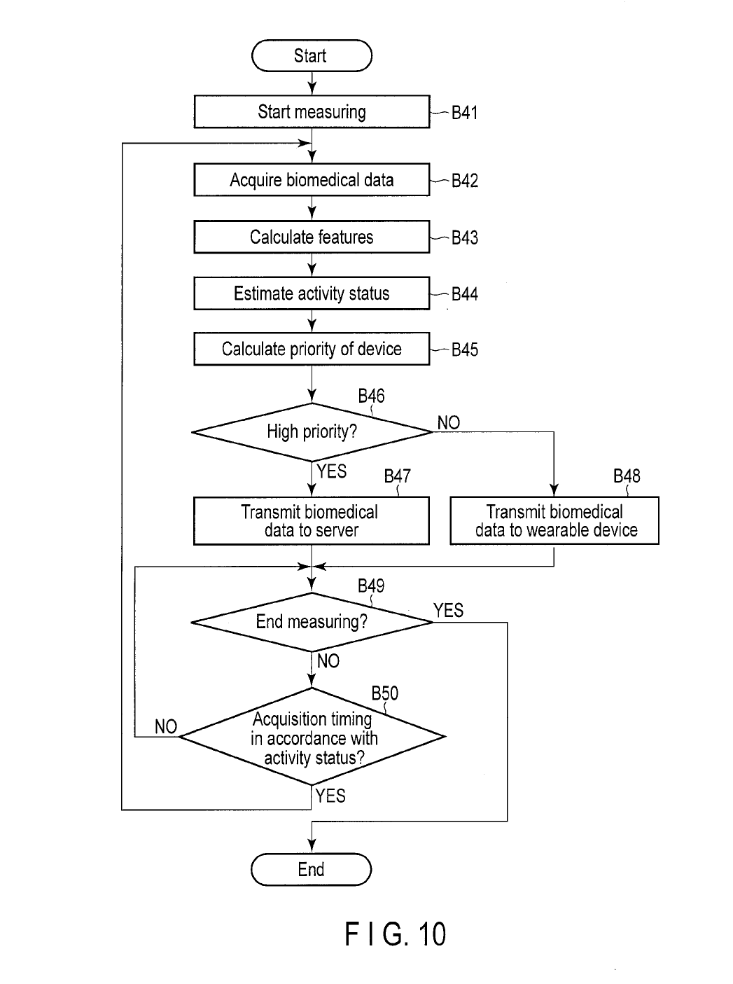

In the following, the processing procedure of the wearable device of the fourth embodiment will be explained with reference to a flowchart of FIG. 10. The processing of the wearable device 41 of transmitting to the server 12 biomedical data acquired in the wearable device 41 will be mainly explained.

To begin with, the processing of blocks B41-B44 corresponding to that of blocks B11-14 shown in FIG. 5 mentioned above is executed. In the following, biomedical data acquired in block B42 is called object biomedical data for explanation.

Next, the transmission controller 412 prioritizes the wearable devices 41-43 based on the processing capacity information retained in the device priority table 411. In this case, the transmission controller 412 calculates the priority of the wearable devices 41-43 (block B45).

The processing capacity information retained in the device priority table 411 includes information such as processing speed of a processor, memory size (buffer size), consumption power, remaining battery capacity and signal intensity. In this case, when, for example, a processing speed is faster, a memory size is bigger and signal intensity is stronger than the other wearable devices 42 and 43, the transmission controller 412 calculates a high priority. On the other hand, when consumption power is bigger and a remaining battery capacity is smaller than the other wearable devices 42 and 43, the transmission controller 412 calculates a low priority. That is, the priority of the wearable device 41 is calculated by weighting in view of the processing capacity of the wearable device 41 and the processing capacity of the other wearable devices 42 and 43. Note that the processing capacity information retained in the device priority table 411 may be set in advance and that a parameter such as remaining battery capacity and signal intensity, which varies according to the use status of the wearable devices 41-43, may be updated via the server 12 or the like.

Next, the transmission controller 412 determines whether or not the priority of the wearable device 41 calculated is higher than that of the wearable devices 42 and 43 (block B46).

When it is determined that the priority of the wearable device 41 is higher than that of the wearable devices 42 and 43 (block B46, YES), the transmission controller 412 transmits to the server 12 biomedical data acquired by the biomedical data acquisition module 201 in block B42 mentioned above (block B47).

On the other hand, when it is determined that the priority of the wearable device 41 is not higher than that of the wearable devices 42 and 43 (block B46, NO), the transmission controller 412 transmits to a wearable device having the highest priority (wearable devices 42 or 43 in this context) biomedical data acquired by the biomedical data acquisition module 201 in block B42 mentioned above (block B48). Note that when the transmission controller 412 transmits biomedical data to a wearable device having the highest priority, the biomedical data is transmitted from the wearable device to the server 12.

When the processing of block B47 or B48 is executed, the processing of blocks B49 and B50 corresponding to that of blocks B16 and B17 shown in FIG. 5 mentioned above is performed.

While it has been explained in FIG. 10 that biomedical data acquired in the wearable device 41 is transmitted to the server 12 when a priority is higher in the wearable device 41 than the other wearable devices 42 and 43, it may also be possible that when, for example, the priority of the wearable device 41 calculated in block B45 mentioned above exceeds a value set in advance, biomedical data acquired in the wearable device 41 is transmitted to the server 12.

Also, while it has been explained in FIG. 10 that biomedical data acquired in the wearable device 41 is transmitted to the other wearable devices 42 or 43 when a priority is not higher in the wearable device 41 than the other wearable devices 42 and 43, it may be possible that the biomedical data is not transmitted but accumulated in a buffer or the like for saving power in the wearable device 41. The biomedical data accumulated in a buffer should be transmitted altogether when the remaining capacity of the buffer runs short. In addition, it may be possible that when data is transmitted to the other wearable devices 42 or 43, features calculated by the features calculator 202 instead of biomedical data is transmitted.

As mentioned above, the fourth embodiment is structured to calculate the priority of the wearable device 41 based on, for example, its performance (processing capacity) and transmit biomedical data to an external apparatus (the server 12 or the other wearable devices) based on the calculated priority. That is, in the fourth embodiment, when, for example, the user wears the plurality of wearable devices 41-43, biomedical data acquired in each of the wearable devices 41-43 is altogether transmitted from a high-priority wearable device to the server 12. This makes it possible to transmit the biomedical data efficiently and therefore to save power in the wearable devices 41-43.

While it has been explained in the fourth embodiment that all the biomedical data acquired in, for example, the wearable device 41 is transmitted to an external apparatus, the fourth embodiment may be structured to calculate the priority of each of the sensors 115a-c included in the sensor module 115 of the wearable device 41 and transmit biomedical data to the server 12 based on the priority of each of the sensors 115a-c, as explained in the third embodiment above.

In the fourth embodiment, since the user wears the plurality of wearable devices 41-43, the position of wearing the wearable devices 41-43 is taken into account for the calculation of the priority of each of the sensors 115a-c. In detail, when the wearable device 41 is a device having a shape such as a watch worn to the wrist of the user and the user's activity status estimated by the activity status estimation module 203 is “Sleeping,” a lower priority is given to the core body temperature sensor 115a and the pulse sensor 115b in which biomedical data varies to a small extent, and a higher priority is given to the acceleration sensor 115c in which biomedical data varies to a great extent. On the other hand, when the wearable device 41 has a shape such as an adhesive plaster worn to the waist of the user and the user's activity status estimated by the activity status estimation module 203 is “Sleeping,” a higher priority is given to the core body temperature sensor 115a and the pulse sensor 115b in which relatively precise biomedical data can be acquired, and a lower priority is given to the acceleration sensor 115c in which biomedical data varies to a small extent.

Thus, by calculating the priority of each of the sensors 115a-c in which the position of wearing the wearable device 41 is taken into account, it is possible to transmit to the server 12 only biomedical data more useful in analysis of the server 12.

In addition, it may also be possible that features calculated by the features calculator 202, not biomedical data, is transmitted to an external apparatus for a low-priority sensor among the sensors 115a-c.

Further, while it has been explained in FIG. 10 that a timing of acquiring biomedical data in accordance with the user's activity status is controlled, the fourth embodiment may be applied when controlling a timing of transmitting biomedical data to an external apparatus in accordance with the user's activity status explained in the first embodiment above.

That is, according to the fourth embodiment, it is possible to save power in the wearable device 41 by controlling the acquisition interval, transmission interval and transmission method (transmitting raw data or features) based on the priority of the above-mentioned device and each of the sensors 115a-c.

Note that while the wearable device 41 has been explained among the wearable devices 41-43 worn by the user in the fourth embodiment, it is possible to save power also in the wearable devices 42 and 43 by executing similar processing (control).

Also, the biomedical data acquired by the same kind of sensor may be transmitted from a plurality of wearable devices to the server 12. In this case, even when biomedical data is not properly acquired in a wearable device, it is possible to avoid loss of biomedical data necessary for analysis in the server 12.

According to at least one of the above-mentioned embodiments, it is possible to save power of a wearable device (electronic apparatus).

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An electronic apparatus comprising a sensor configured to detect biomedical data, comprising:
an acquisition controller configured to acquire first and second biomedical data detected by the sensor;
an estimation controller configured to estimate status based on the first biomedical data; and
a transmission controller configured to transmit the second biomedical data to an external apparatus at a first time based upon the estimated status.

2. The electronic apparatus of claim 1, wherein the first biomedical data and the second biomedical data are identical.

3. The electronic apparatus of claim 1, further comprising a calculator configured to calculate a priority of the sensor based on the estimated status in accordance with a communication status with the external apparatus,
wherein the transmission controller is configured to transmit the second biomedical data to the external apparatus when the calculated priority of the sensor exceeds a value set in advance.

4. The electronic apparatus of claim 1, further comprising a calculator configured to calculate a priority of the electronic apparatus based on a performance of the electronic apparatus,
wherein the transmission controller is configured to transmit the second biomedical data to the external apparatus based on the calculated priority of the electronic apparatus.

5. An electronic apparatus comprising a sensor configured to detect biomedical data, comprising:
an acquisition controller configured to acquire first and second biomedical data detected by the sensor;
an estimation controller configured to estimate status based on the first biomedical data; and
a transmission controller configured to transmit the second biomedical data to an external apparatus,
wherein the acquisition controller is configured to acquire the first and second biomedical data at a first time based on the estimated status.

6. The electronic apparatus of claim 5, wherein the first biomedical data and the second biomedical data are identical.

7. The electronic apparatus of claim 5, further comprising a calculator configured to calculate a priority of the sensor based on the estimated status in accordance with a communication status with the external apparatus,
wherein the transmission controller is configured to transmit the second biomedical data to the external apparatus when the calculated priority of the sensor exceeds a value set in advance.

8. The electronic apparatus of claim 5, further comprising a calculator configured to calculate a priority of the electronic apparatus based on a performance of the electronic apparatus,
wherein the transmission controller is configured to transmit the second biomedical data to the external apparatus based on the calculated priority of the electronic apparatus.

9. A communication control method, comprising:
acquiring first and second biomedical data detected by a sensor comprised in an electronic apparatus;
estimating status based on the first biomedical data; and
transmitting the second biomedical data to an external apparatus at a first time based on the estimated status.

10. A communication control method, comprising:
acquiring first and second biomedical data detected by a sensor in an electronic apparatus;
estimating status based on the first biomedical data; and
transmitting the second biomedical data to an external apparatus,
wherein the acquiring comprises acquiring the first and second biomedical data at a timing in accordance with the estimated status.
